(19) United States
(12) Patent Application Publication
(10) Pub. No.: US 2010/0076404 A1
(43) Pub. Date: Mar. 25, 2010

(54) SUBARACHNOID CATHETERS CONFIGURED TO FACILITATE CIRCULATORY FLUID FLOW

(76) Inventor: Lawrence Scott Ring, Valencia, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/234,348

(22) Filed: Sep. 19, 2008

(51) Int. Cl. A61M 25/00

(52) U.S. Cl. 604/523

(57) ABSTRACT

Catheters, with an internal lumen and distal apertures, that are configured such that physiological factors will cause bodily fluid to move in an out of the distal apertures and the portion of internal lumen adjacent to the apertures, as well as axially and/or angularly along the outer surface of the catheter adjacent to the apertures.

### BACKGROUND

1. Field of Inventions

The present inventions relate generally to catheters that may be used to, for example, deliver medication to the subarachnoid space.

2. Description of the Related Art

Implantable infusion devices have been used to provide patients with a medication or other substance (collectively “infusible substance”) and frequently include an implantable pump and a catheter. A reservoir stores the infusible substance within the pump and, in some instances, implantable pumps are provided with a fill port that allows the reservoir to be transcutaneously filled (and/or re-filled) with a hypodermic needle. The reservoir is coupled to a fluid transfer device within the pump which is, in turn, connected to an outlet port. The catheter, which has one or more outlets, may be connected to the outlet port. As such, the infusible substance may be transferred from the reservoir to the target body region by way of the fluid transfer device and catheter.

One issue associated with the delivery of infusible substance into the subarachnoid space around the spinal cord or brain is the prolonged exposure of the arachnoid mater and adjacent tissues to high concentration drugs at or near the catheter outlets. Prolonged exposure of the arachnoid mater to high concentration drugs may result in irritation of the arachnoid mater and adjacent tissues (e.g. pia mater) that, in turn, may lead to granuloma formation. For example, in those instances where an aperture directly faces and/or is in contact with the arachnoid mater for a prolonged period, the arachnoid mater and adjacent tissues will be exposed to the high concentration drug within the aperture and adjacent regions of the internal lumen for the prolonged period. Granulomas may partially or completely block the outlets, thereby preventing the patient from receiving the intended dosage of infusible substance. Additionally, in the specific context of delivery to the subarachnoid space around the spinal cord, the formation of granulomas may lead to spinal cord compression.

### SUMMARY

Catheters in accordance with various implementations of at least some of the present inventions include distal apertures and distal structures that are configured such that physiological factors will cause bodily fluid to move in and out of the distal apertures and the portion of internal lumen adjacent to the apertures, as well as axially and/or angularly along the outer surface of the catheter adjacent to the apertures. Such movement of bodily fluid tends to reduce the concentration of infusible substance (e.g. high concentration drugs) to which adjacent tissue may be exposed. In the exemplary context of the subarachnoid space around the spinal cord or brain, such movement of cerebrospinal fluid reduces the concentration of drugs to which the arachnoid mater and adjacent tissues (e.g. pia mater) may be exposed for prolonged periods, thereby reducing the likelihood of granuloma formation.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a representation of an implantable infusion device with a catheter that is located in the subarachnoid space in accordance with one embodiment of a present invention.

FIG. 2 is a section view of a catheter that is located within the subarachnoid space.

FIG. 3 is a perspective view of a catheter in accordance with one embodiment of a present invention.

FIG. 4 is a section view taken along line 4-4 in FIG. 3.

FIG. 5 is another section view taken along line 4-4 in FIG. 3.

FIG. 6 is a section view of the catheter illustrated in FIGS. 3-5 positioned in the subarachnoid space.

FIG. 7 is a section view of a catheter in accordance with one embodiment of a present invention.

FIG. 8 is a perspective view of a catheter in accordance with one embodiment of a present invention.

FIG. 9 is another perspective view of the catheter illustrated in FIG. 8.

FIG. 10 is a perspective view of a catheter in accordance with one embodiment of a present invention.

FIG. 11 is another perspective view of the catheter illustrated in FIG. 10.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion devices described herein and, instead, are applicable to other implantable or otherwise ambulatory infusion devices that currently exist or are yet to be developed.

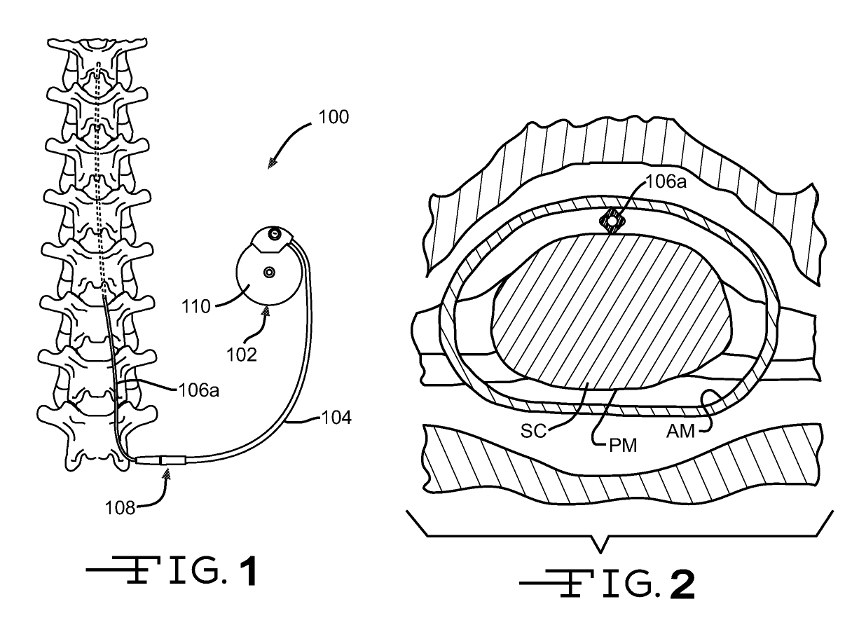

One example of an implantable infusion device in accordance with a present invention is generally represented by reference numeral 100 in FIG. 1. The implantable infusion device 100 includes an implantable pump 102, a proximal catheter 104 that is connected to the pump, a subarachnoid catheter 106a with an internal lumen and plurality of distal apertures, and a connector assembly 108. The implantable pump 102 includes a housing 110. An infusible substance reservoir, a fluid transfer device, control electronics and various other devices are carried within the housing 110. Although the present inventions are not limited to any particular type of implantable pump, exemplary pumps are described in U.S. Patent Pub. Nos. 2005/0273083 and 2006/0270983, which are incorporated herein by reference. The connector assembly 108 may be used to connect the proximal catheter 104 to the subarachnoid catheter 106a after the subarachnoid catheter has been positioned within the patient's body. For example, in those instances where a stylet is used to push the distal portion of the subarachnoid catheter 106a to the target location, the subarachnoid catheter will be connected to the proximal catheter 104 after the stylet has been removed. The infusible substance may then be delivered to, for example, the portion of the subarachnoid space along the spine between the pia mater PM around the spinal cord SC and the arachnoid mater AM, as is illustrated in FIG. 2. In other implementations, the connector assembly 108 may be omitted and the catheter 106a connected directly to the implantable pump 102 before or after the catheter has been positioned.

As is discussed in greater detail below, the present subarachnoid catheters may be configured in such a manner that physiological factors (e.g. movement of the spine or beating of the heart) will cause cerebrospinal fluid (CSF) to move in and out of the distal apertures and the portion of internal lumen adjacent to the apertures, as well as axially and/or angularly along the outer surface of the catheter adjacent to the apertures, thereby reducing the concentration of infusible substance (e.g. high concentration drugs) to which the arachnoid mater and adjacent tissues (e.g. the pia mater) may be exposed for prolonged periods. For example, the present subarachnoid catheters may include channels and/or protrusions and/or projecting members that are associated with the distal portion apertures and facilitate the above-described movement of CSF.

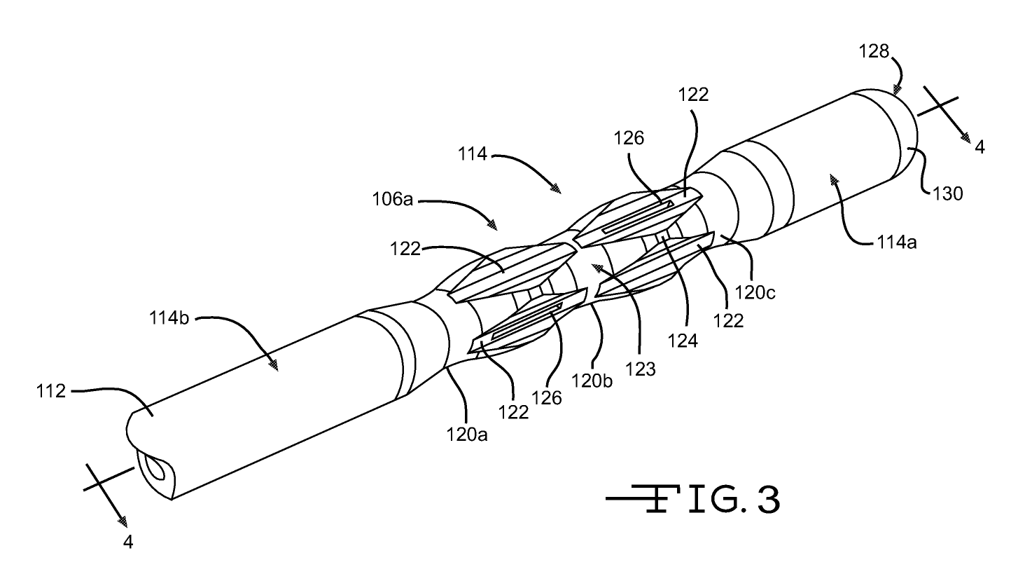

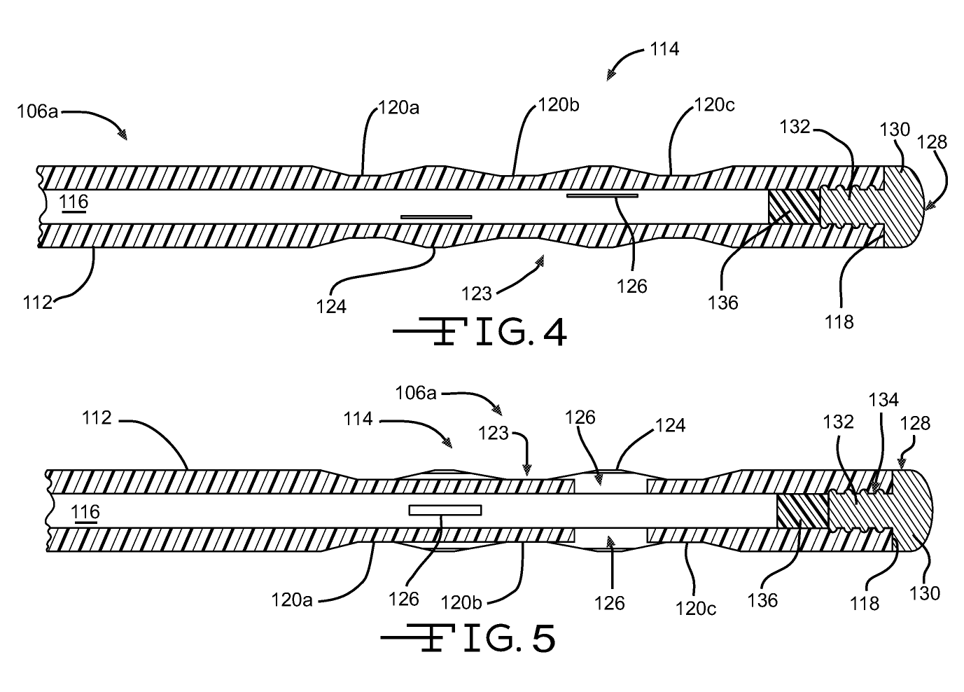

Turning to FIGS. 3-5, the exemplary subarachnoid catheter 106a includes a catheter body 112 with a distal portion 114 and a central lumen 116 that extends from the proximal end of the catheter (i.e. the end adjacent to the connector assembly 108 in FIG. 1) to the distal end 118 of the catheter. The catheter distal portion 114 includes a plurality of exterior flow regions 120a-c which have a perimeter, i.e. a circumference in the illustrated embodiment, that is smaller than that of adjacent regions of the distal portion. A plurality of slots 122 are located between the flow regions 120a-c, as are a plurality of protrusions 124. The distal portion 114 also includes a plurality of apertures 126 that extend through the catheter wall, from the exterior of the distal portion to the central lumen 116, and are located in some of the slots 122.

In the exemplary embodiment illustrated in FIGS. 3-5, the apertures 126 are rectangular in shape and are narrower than the slots 122. There are four slots 122, four protrusions 124 and two diametrically opposed apertures 126 located between the flow regions 120a and 120b as well as four slots, four protrusions and two diametrically opposed apertures between the flow regions 120b and 120c in the exemplary embodiment. The apertures 126 between the flow regions 120a and 120b are offset from apertures between flow regions 120b and 120c by ninety degrees. Accordingly, the apertures 126 between the flow regions 120a and 120b are axially and angularly offset from the apertures 126 between the flow regions 120b and 120c. As used herein, two things are “axially offset” if they are not aligned with the same portions of the longitudinal axis of the catheter. For example, the apertures 126 visible in FIG. 5 between flow regions 120b and 120c are axially aligned with one another, and are axially offset from the aperture 126 visible in FIG. 5 between flow regions 120a and 120b. As used herein, two things are “angularly offset” if, regardless of axial alignment or cross-sectional shape of the catheter, they are offset about the longitudinal of the catheter. For example, the aperture 126 visible in FIG. 3 between flow regions 120a and 120b is angularly offset from the aperture 126 visible in FIG. 3 between flow regions 120b and 120c by 90 degrees.

It should be noted here that the shape, number, spacing, axial location, and/or angular offset of the apertures 126 may be varied as desired, as may the shape, number and/or location of the flow regions 120a-c and slots 122. By way of example, but not limitation, the apertures 126 may be circular in shape and/or may be located in the flow regions 120a-c instead of the slots 122.

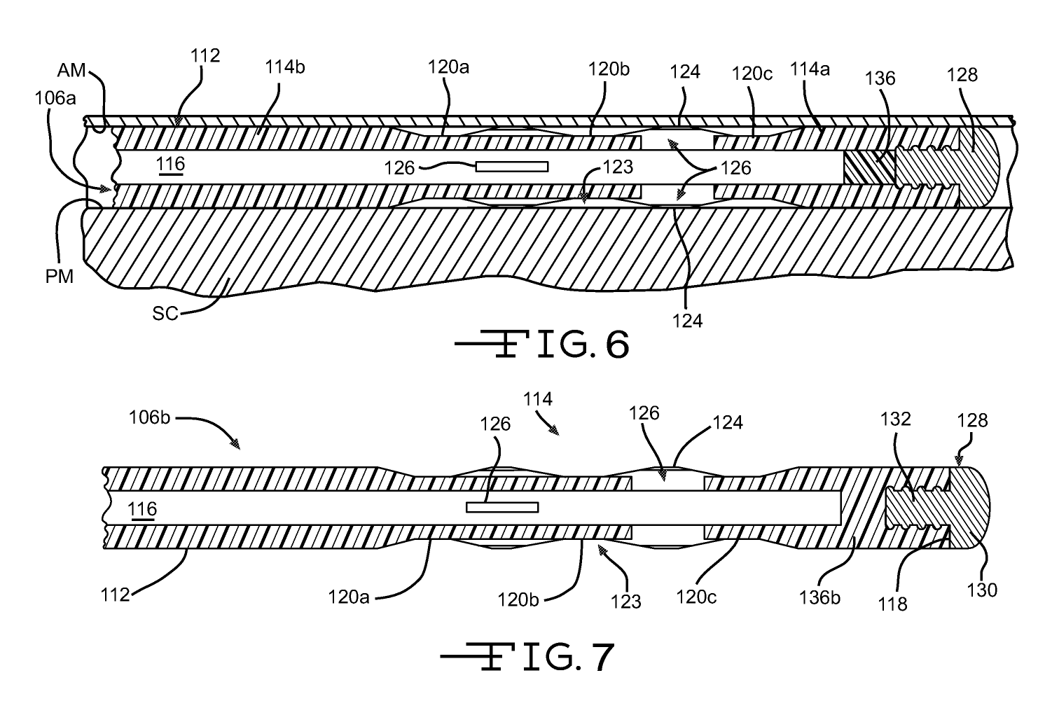

The flow regions 120a-c and slots 122 together define channels 123 (FIG. 3) which extend to and from, among other things, apertures 126 that are axially and angularly offset from one another so that fluid can flow along the outer surface of catheter distal portion 114 from one such aperture to the other. The channels 123 also facilitate fluid flow around the protrusions 124 and over the outer surface of the region within the distal portion 114 that extends from the proximal end of the flow region 120a to the distal end of the flow region 120c. Accordingly, and turning to FIG. 6, when the distal portion 114 of the catheter 106a is positioned between the spinal cord SC and the arachnoid mater AM, the distal portion regions 114a and 114b will be in contact with tissue. Some or all (as shown) of the protrusions 124, which are located between the distal portion regions 114a and 114b, will also be in contact with tissue. No matter how the catheter 106a is rotationally oriented relative to the spinal cord, CSF will be free to flow along the exterior of the catheter distal portion 114 from one flow region 120a-c to another (sometimes referred to herein as “axial flow”), in and out of the apertures 126 (sometimes referred to herein as “radial flow”), and around the perimeter of the distal portion 114 (sometimes referred to herein as “angular flow”) while the distal portion regions 114a and 114b are in contact with tissue. The axial, radial and angular flow of CSF, which is the result of physiological factors (e.g. the movement of the spine and beating of the heart), dilutes medication within the lumen 116, the apertures 126 and along the outer surface of the catheter distal portion 114 that may be in contact with the arachnoid mater for prolong periods. Thus, the configuration of the distal portion 114 reduces the likelihood that granulomas, which may be due to prolonged exposure of the arachnoid mater and adjacent tissues to high concentration drugs, will form. There may also be a clinical benefit associated with more uniform distribution of the drugs.

A marker tip 128 is carried on the distal end 118 of the catheter body 112. The exemplary marker tip 128 is radiopaque and includes a main portion 130 and a connector 132. The connector 132, which is located within the central lumen 116, has a plurality of indentations 134 such as, for example, the illustrated plurality of longitudinally spaced concentric grooves. The catheter distal portion 114 may be heated to its melting point after the marker tip connector 132 has been inserted into the central lumen 116 so that catheter material will flow into the indentations 134. A mandrel (not shown) will also be inserted into the central lumen 116 proximal to the marker tip 128 prior to heating. The catheter material within the indentations 134, once cooled, secures the marker tip 128 to the catheter body 112. In other implementations, the connector may be smooth and secured to the catheter distal portion 114 with an adhesive. In still other implementations, marker tips may be configured such that they can be mounted on the catheter body distal end 118, and cover the distal end of the central lumen 116, without a connector that extends into the central lumen.

The exemplary subarachnoid catheter 106a illustrated in FIGS. 3-5 is also provided with an abutment 136 that is located within the central lumen 116 proximal to the marker tip 128. The exemplary abutment 136, which is cylindrical in shape and has an outer diameter (OD) that is equal to the inner diameter (ID) of the catheter body 112, may be formed from any suitable material and secured to the catheter distal portion 114 with an adhesive. The abutment 136 prevents stylets from separating the marker tip 128 from the distal end 118 of the catheter body 112 as the stylet is pushing the distal portion 114 of the catheter 106a to a target location within, for example, the subarachnoid space around the spinal cord.

The abutment may, alternatively, be an integral portion of the catheter body 112. The subarachnoid catheter 106b illustrated in FIG. 7 is substantially similar to subarachnoid catheter 106a and similar elements are represented by similar reference numerals. Here, however, the abutment 136b is an integral part of the catheter body distal portion 114 as opposed to a separate structure that is secured to the distal portion with, for example, an adhesive. The integral abutment 136b, which performs the same functions as abutment 136, may be formed in a variety of ways. For example, a donor structure (not shown), such as a cylindrical donor structure that is formed from the same material as the catheter body 112 and has an OD that corresponds to the ID of the catheter body, may be inserted into the lumen 116. The catheter body 112 and donor structure may then be heated to the melting temperature of the material. A mandrel may also be inserted into the central lumen 116 proximal to the donor structure prior to heating. The catheter body distal portion 114 and donor structure will merge and, once cooled, will provide the catheter body distal portion with the integral abutment 136b illustrated in FIG. 7. In those instances where the marker tip 128 is secured to the catheter body 112 by heating the catheter distal portion 114 to its melting point after the marker tip connector 132 has been inserted into the central lumen 116, the marker tip may be secured to the catheter body while the integral abutment is being formed.

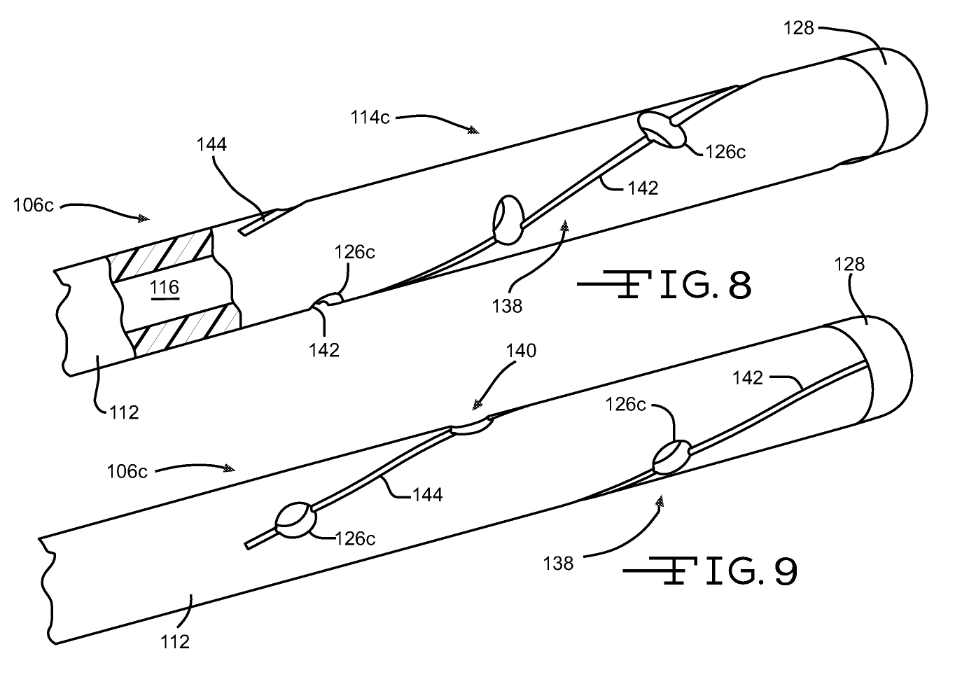

Another exemplary catheter is generally represented by reference numeral 106c in FIGS. 8 and 9. The exemplary catheter 106c includes a catheter body 112 with a distal portion 114c, a central lumen 116 and a plurality of apertures 126c that extend from the exterior of the distal portion to the central lumen. The exemplary catheter 106c may include a marker tip 128, and may include an internal abutment (not shown) such as the abutment 136 or the abutment 136a, as are described above.

Some or all of the apertures may be axially and angularly offset from one another in order to insure that at least some of the apertures will be exposed to CSF within the subarachnoid space should other apertures be directly facing, and/or blocked by, the spinal cord or the arachnoid mater. Although not limited to any particular number or orientation, there are six apertures 126c in the illustrated embodiment and the apertures are arranged in a first set 138 of three apertures and a second set 140 of three apertures. Adjacent apertures 126c in each set 138 and 140 are axially offset and angularly offset by 45 degrees. The first and second sets 138 and 140 are axially aligned (i.e. the distal-most apertures 126c are axially aligned, the middle apertures are axially aligned, and the proximal-most apertures are axially aligned) and are angularly offset from one another by 180 degrees.

The exemplary catheter 106c also includes first and second channels 142 and 144 that project inwardly from the outer surface of the catheter distal portion 114c. As used herein, a surface “projects inwardly” if the radial distance from the longitudinal axis to the surface is less than the radial distance from the longitudinal axis to adjacent surfaces. The first channel 142 connects, and provides a fluid path between, the apertures 126c in the first set 138 to one another and extends proximally and distally beyond the first set. Similarly, the second channel 144 connects, and provides a fluid path between, the apertures 126c in the second set 140 to one another and extends proximally and distally beyond the second set. The channels 142 and 144 are also spiral shaped and, accordingly, the distal ends of the channels are axially and angularly offset from the distal-most apertures 126c and the proximal ends of the channels are axially and angularly offset from the proximal-most apertures.

No matter how the catheter 106c is rotationally oriented relative to the spinal cord, CSF will be free to flow axially and angularly along the first and second channels 142 and 144 as well as axially and angularly along the outer surfaces of the distal portion 114c that are not in contact with tissue. CSF will also be free to flow radially in and out of the apertures 126c while the distal portion 1 14c is in contact with tissue. The radial flow may be directly in and out of the subarachnoid space or, in those instances where an aperture 126c is in contact with tissue, in and out the subarachnoid space by way of the associated channel 142 or 144. Such flow of CSF, which is the result of physiological factors (e.g. the movement of the spine and beating of the heart), dilutes medication within the lumen 116, the apertures 126c and on the surfaces of the catheter distal portion 114c adjacent to the apertures that may be in contact with the arachnoid mater for prolong periods. Thus, the configuration of the distal portion 114c reduces the likelihood that granulomas, which may be due to prolonged exposure of the arachnoid mater and adjacent tissues to high concentration drugs, will form.

It should be noted here that the shape, number, spacing, axial location, and/or angular offset of the apertures 126c may be varied as desired, as may the shape, number and location of the channels 142 and 144. By way of example, but not limitation, the first and second sets may be both axially and angularly offset. Alternatively, the apertures 126c may be arranged in four sets that are angularly offset from one another by 90 degrees and, within each set, the apertures are axially offset and angularly aligned. Here, the four channels that connect the apertures in each set will extend axially. Moreover, in some implementations, the channels may be replaced by slits that extend completely through the catheter wall.

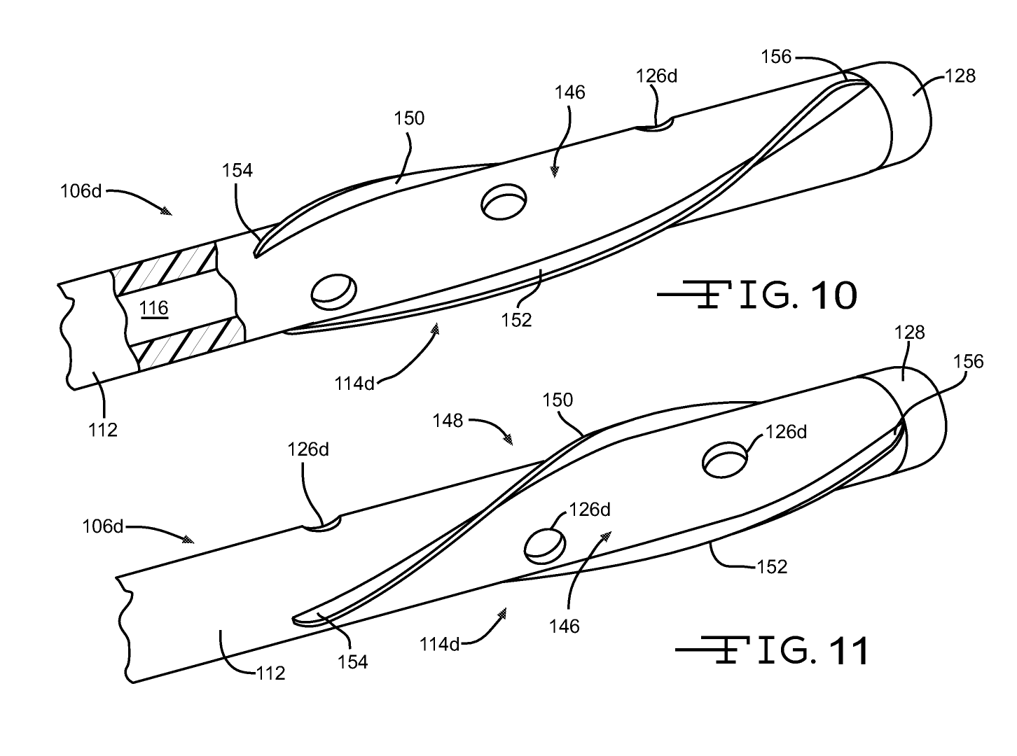

Other exemplary catheters include a plurality of distal portion apertures and outwardly projecting members adjacent to the apertures. One example of such a catheter is generally represented by reference numeral 106d in FIGS. 10 and 11. The exemplary catheter 106d includes a catheter body 112 with a distal portion 114d, a central lumen 116 and a plurality of apertures 126d that extend from the exterior of the distal portion to the central lumen. The exemplary catheter 106d may include a marker tip 128, and may include an internal abutment (not shown) such as the abutment 136 or the abutment 136a, as are described above.

The apertures 126d in the illustrated embodiment are axially and angularly offset from one another. Although not limited to any particular number or orientation, there are six apertures 126d in the illustrated embodiment and the apertures are arranged in first set 146 of three apertures and a second set 148 of three apertures. Adjacent apertures 126d in each set 146 and 148 are axially offset and angularly offset by 45 degrees. The first and second aperture sets 146 and 148 are longitudinally aligned (i.e. the distal-most apertures 126d are axially aligned, the middle apertures are axially aligned, and the proximal-most apertures are axially aligned) and are angularly offset from one another by 180 degrees.

The exemplary catheter 106d also includes a pair of outwardly projecting members 150 and 152 that are positioned between the first and second sets 146 and 148 of apertures 126d. In the illustrated embodiment, the outwardly projecting members 150 and 152 are spiral shaped, angularly offset from one another by 180 degrees (at each axially aligned point) and are angularly offset from the apertures 126d by 90 degrees. The outwardly projecting members 150 and 152, which each include tapered proximal and distal ends 154 and 156, extend axially and angularly beyond the first and second aperture sets 146 and 148 in the proximal and distal directions. In some implementations, the outwardly projecting members 150 and 152 extend the entire length of the catheter. The exemplary outwardly projecting members 150 and 152 are also configured such that they will deflect and lay flat against the catheter body 112 during implantation through, for example, an insertion needle and then spring back to the illustrated orientation when deployed in the subarachnoid space.

Portions of the projecting members 150 and 152 will engage tissue when the catheter 106d is deployed in the subarachnoid space. As a result, the apertures 126d will not be blocked by the arachnoid mater or spinal cord. No matter how the catheter 106d is rotationally oriented relative to the spinal cord, CSF will be free to flow axially and angularly along the outer surface of the distal portion 114d, although the fluid may no be able to cross portions of a projecting member that are in contact with tissue. CSF will also be free to flow in and out of the apertures 126d. Such flow of CSF, which is the result of physiological factors (e.g. the movement of the spine and beating of the heart), dilutes medication within the lumen 116, the apertures 126d and on the exterior of the distal portion 1 14d that may be adjacent to the arachnoid mater for prolong periods. Thus, the configuration of the distal portion 114d reduces the likelihood that granulomas, which may be due to prolonged exposure of the arachnoid mater and adjacent tissues to high concentration drugs, will form.

With respect to materials, suitable materials for the catheter body 112 include, but are not limited to polymers such as polyurethane (e.g. Carbothane® 95A), silicone, polyethylene, and polypropylene. In addition to having higher tensile strength and tear resistance than, for example, silicone, the Carbothane® 95A is also more lubricious. The additional lubricity may reduce the irritation to the arachnoid mater associated with the presence of the catheter and, accordingly, further reduce the likelihood of granuloma formation. Suitable materials for the marker tip 128 include, but are not limited to, radiopaque materials such as platinum, gold, tungsten, barium filled plastics, and iridium.

With respect to dimensions, the exemplary catheter body 112, which is configured for use in the subarachnoid space, is circular in cross-section and has an OD of about 0.055 inches and an ID of about 0.021 inches. The present catheters are not, however, limited to a circular cross-sectional shape. Regardless of the exterior shape, the shape of the lumen 116 may be circular, as shown, or may be a shape that helps prevent kinks and occlusions (e.g. star or triangular in shape). The length of the catheter body 112 may also vary from about 10 inches to about 40 inches, depending on the intended application.

As for the specifics of the exemplary catheters 106a and 106b, the OD at the exterior flow regions 120a-c is about 0.042 inches, and adjacent exterior flow regions are about 0.120 inch apart. Turning to exemplary catheter 106c, the diameter of the apertures 126c is about 0.015 inches, the longitudinal distance between adjacent apertures in each set is about 0.120 includes. The channels 142 and 144 are about 0.005 inches wide, 0.005 inches deep and extend distally about 0.060 inches beyond the distal-most apertures 126c and proximally about 0.060 inches beyond the proximal-most apertures. With respect to the catheter 106d, the diameter of the apertures 126d is about 0.015 inches, and the projecting members 150 and 152 are about 0.010 inches high (measured from the outer surface of the catheter body 12) and about 0.005 inches thick.

Turning to the formation of the various structures that facilitate CSF flow and drug dilution, the flow regions 120a-c, slots 122, protrusions 124, channels 142 and 144, and projective members 150 and 152 may be thermal formed over a mandrel (or formed by some other secondary forming operation) after the catheter body has been extruded. The apertures 126-126d may be formed thereafter. The portion of the catheter which includes the structures that facilitate CSF flow may also be formed separately (e.g. by injection molding) and butt-spliced onto a catheter tube.

Although the inventions disclosed herein have been described in terms of the preferred embodiments above, numerous modifications and/or additions to the above-described preferred embodiments would be readily apparent to one skilled in the art. By way of example, but not limitation, the present inventions are applicable to catheters that supply stimulation energy, as opposed to or in addition to, infusible substances. Such catheters are sometimes referred to a spinal cord stimulation leads. It is intended that the scope of the present inventions extend to all such modifications and/or additions and that the scope of the present inventions is limited solely by the claims set forth below.

I claim:

1. A catheter, comprising:
a tubular catheter body including a wall, a distal portion and a lumen that extends to the distal portion;
a plurality of apertures that extend through the wall of the distal portion to the lumen, at least two of the apertures being both axially and angularly offset from one another; and
at least one channel which fluidly connects the at least two apertures that are both axially and angularly offset.

2. A catheter as claimed in claim 1, wherein the at least two apertures that are both axially and angularly offset comprise three apertures that are both axially and angularly offset.

3. A catheter as claimed in claim 1, wherein the at least one channel extends axially and angularly beyond the at least two axially and angularly offset apertures in at least one of the proximal direction and the distal direction.

4. A catheter as claimed in claim 1, wherein
the at least two apertures comprise a first set of at least two apertures that are both axially and angularly offset and a second set of at least two apertures that are both axially and angularly offset, the second set being angularly offset from the first set; and
the at least one channel comprises a first channel which fluidly connects the at least two apertures in the first set and a second channel which fluidly connects the at least two apertures in the second set.

5. A catheter as claimed in claim 1, wherein the at least two apertures each define an aperture width and the channel defines a width that is less than the aperture width.

6. A catheter as claimed in claim 1, wherein the at least two apertures each define an aperture width and the channel defines a width that is greater than the aperture width.

7. A catheter as claimed in claim 1, wherein
the distal portion includes a plurality of axially offset flow regions that extend around the perimeter a plurality of slots that extend between the flow regions; and
the at least one channel is defined by portions of the flow regions and the slots.

8. A catheter as claimed in claim 7, further comprising:
a plurality of protrusions between the flow regions and angularly offset from the slots.

9. A catheter, comprising:
a tubular catheter body including a wall, a distal portion, an outer surface and a lumen that extends to the distal portion;
a plurality of apertures that extend through the wall of the distal portion to the lumen, at least two of the apertures being both axially and angularly offset from one another; and
at least one spiral shaped member projecting outwardly from the outer surface of the distal portion.

10. A catheter as claimed in claim 9, wherein the at least two apertures that are both axially and angularly offset comprise three apertures that are both axially and angularly offset.

11. A catheter as claimed in claim 9, wherein the at least one spiral shaped member extends axially and angularly beyond the at least two axially and angularly offset apertures in at least one of the proximal direction and the distal direction.

12. A catheter as claimed in claim 9, wherein
the at least two apertures comprise a first set of at least two apertures that are both axially and angularly offset and a second set of at least two apertures that are both axially and angularly offset, the second set being angularly offset from the first set; and
the at least one spiral shaped member comprises first and second spiral shaped members that are angularly offset from one another.

13. A catheter as claimed in claim 9, wherein the at least one spiral shaped member defines tapered proximal and distal ends.

14. A catheter for providing drugs to the subarachnoid space between the brain or spinal cord and the arachnoid mater, the subarachnoid space including cerebrospinal fluid, the catheter comprising:
a tubular catheter body including a distal portion and a lumen that extends to the distal portion;
a plurality of axially and angularly offset apertures that extend through the distal portion to the lumen; and
means, associated with the distal portion of the catheter body, for facilitating the axial, radial and angular flow of drugs and cerebrospinal fluid relative to the catheter distal portion in response to physiological factors.

15. A catheter as claimed in claim 14, wherein the plurality of apertures comprise a first set of at least two apertures that are both axially and angularly offset and a second set of at least two apertures that are both axially and angularly offset, the second set being angularly offset from the first set.

16. A catheter, comprising:
a tubular catheter body including a distal portion, a distal end and a lumen that extends to the distal portion, the distal portion defining first, second and third axially offset distal portion regions, the second distal portion region extending to the distal end, the third distal portion region being located between the first and second distal portion regions, and the first and second distal portion regions having circumferences of substantially the same size;
a plurality of apertures that extend through the distal portion to the lumen within the third distal portion region; and
a plurality of indentations and protrusions located within the third distal portion region.

17. A catheter as claimed in claim 16, wherein the plurality of apertures comprise a first set of at least two apertures that are both axially and angularly offset and a second set of at least two apertures that are both axially and angularly offset, the second set being angularly offset from the first set.
